(19) United States
(12) Patent Application Publication
(10) Pub. No.: US 2008/0119637 A1
(43) Pub. Date: May 22, 2008

(54) Pipette tip column, resin and method of use for extracting an analyte

(76) Inventors: Douglas T. Gjerde, Saratoga, CA (US); Liem Nguyen, San Jose, CA (US); Jeremy Lambert, Worcester, MA (US)

Correspondence Address: PHYNEXUS, INC., 3670 CHARTER PARK DRIVE, SAN JOSE, CA, 95136, US

(21) Appl. No.: 11/986,335

(22) Filed: Nov. 20, 2007

Related U.S. Application Data
(60) Provisional application No. 60/860,636, filed on Nov. 21, 2006.

(51) Int. Cl. C07K 1/02

(52) U.S. Cl. 530/334

(57) ABSTRACT

The invention provides extraction columns for the purification of an analyte (e.g., a biological macromolecule, such as a polypeptide or protein) from a sample solution, as well as methods for making and using such columns. The columns typically include a bed of extraction medium in a column. In some embodiments, the extraction columns employ modified pipette tips as column bodies. In some embodiments, the invention provides resins and methods that facilitate elution of analyte in a small volume of liquid. In some embodiments, the analyte is an active protein.

### CROSS REFERENCE TO RELATED APPLICATIONS

This application claims priority to and benefit of U.S. Provisional Patent Application No. 60/860,636 filed Nov. 21, 2006; U.S. patent application Ser. No. 10/434,713 filed May 8, 2003 and U.S. patent application Ser. No. 10/620,155, filed Jul. 14, 2003, the disclosures of which are incorporated herein by reference in their entirety for all purposes.

### FIELD OF THE INVENTION

This invention relates to improved resins in a pipette tip column and methods for extracting an analyte from a sample solution. The analytes can include biomolecules, particularly biological macromolecules such as proteins. The devices and methods of this invention are particularly useful in the biological sciences for sample preparation and analysis with analytical technologies employing biochips, mass spectrometry and a variety of instrumentation and biological assays.

### BACKGROUND OF THE INVENTION

Solid phase extraction is a powerful technology for purifying and concentrating analytes, including biomolecules. For example, it is one of the primary tools used for preparing protein samples prior to analysis by any of a variety of analytical techniques, including mass spectrometry, surface plasmon resonance, nuclear magnetic resonance, x-ray crystallography, and the like. With these techniques, typically only a small volume of sample is required. However, it is often critical that interfering contaminants be removed from the sample and that the analyte of interest is present at some minimum concentration. Thus, sample preparation methods are needed that permit the purification and concentration of small volume samples with minimal sample loss.

The subject invention involves methods and devices for extracting an analyte from a sample solution using a packed bed of extraction medium, e.g., a bed of gel-type beads derivatized with a group having an affinity for IgG or a recombinant protein. These methods, and the related devices and reagents, will be of particular interest to the life scientist, since they provide a powerful technology for purifying, concentrating and analyzing active proteins.

The methods of the instant invention typically involve a capture step, an optional wash and an elution. The capture is performed by passing a sample containing an analyte of interest through a packed bed of extraction medium. As the sample passes through the bed, the analytes in the sample are retained by the extraction medium while the sample solution, salts, and other impurities pass through. After the sample is captured, the bed of medium is optionally washed with water, buffer or solvent to further remove impurities. Then, the purified analyte is eluted with an appropriate desorption solution.

Using the columns and methods of the instant invention it is possible to obtain highly concentrated purified analyte by eluting the purified analyte in a very small volume. However, certain characteristics of the extraction resin can hinder effective elution of analytes in a small volume. Solid phase extraction resins used for the separation or purification of biomolecules can contain ion exchange groups that are left over from the resin synthesis process. In some cases, residual ion exchange sites are present because they were not reacted completely during the resin synthesis process. In other cases, residual ion exchange sites are artifacts from the substrate or the functional groups. Usually, these residual ion exchange sites do not interfere with the capture of a biomolecule however they can negatively impact efficient analyte elution, particularly elution in a small volume, and especially when it is desirable to keep the analyte active. The instant invention provides methods, resins and columns (such as pipette tip columns) that lack these residual ion exchange sites thus allowing good recovery of active biomolecule analyte in a small volume.

For example, a Protein A agarose affinity resin may contain ion exchange sites that are left over from the resin synthesis. The attachment of the protein A molecule to the agarose substrate is performed in such a way that anion exchange sites remain present after the synthesis is complete (FIG. 2A). Some of these residual anion exchange sites can be associated with Protein A (labelled “IA”) while others are not. These residual anion exchange sites do not interfere with the capture of an antibody protein. But they do negatively impact the efficacy of the elution process as described below.

Capture of an antibody on a Protein A resin column is usually performed at pH 7.4. Antibody protein (labeled “P”) is captured on both the resin surface and the interior of the bead (“AP” in FIG. 2B). In some cases, antibody proteins are captured on Protein A sites that are associated with a residual anion exchange group (labeled “IAP”). After capture the bead is washed with a buffer of the same pH (7.4) to remove non-specifically bound material. Next, the column is optionally washed with saline to remove the buffering capacity of any remaining wash solution. If the buffering capacity remained from the first wash solution, then the low pH desorption solution would first have to neutralize the buffer before the acid could be applied to removing the protein from the column. The second wash removes this buffering capacity thus preparing the column for elution. The captured protein is eluted with the low pH desorption solution and remains active. Next, a base or buffer is added to the protein to raise the pH, keeping the protein active.

But because the Protein A resin also contains residual ion exchange sites, these sites will take up a low pH eluting ion (such as the hydrogen ion in FIG. 2C). There is an ionic threshold must be satisfied before the low pH desorption solution can be applied exclusively to eluting the antibody from the Protein A functional group. The ionic threshold is defined herein as the concentration of the eluting ion that must be reached before the purified analyte is released from the medium. Once the threshold is reached, the hydrogen ions will exclusively protonate the Protein A group, releasing the antibody.

The concentration of these residual ion exchange sites can be extremely high; in some cases, up to 2 M. As a result, a higher ionic threshold must be reached before the antibody is released. That is, more acid is required to get good recovery of the purified antibody. The amount of acid can be increased by increasing the volume or lowering the pH of the eluent, neither of which is optimal, particularly when an active protein in small elution volume is desired.

The resins and columns of the instant invention lack residual ion exchange sites eliminating the need to reach an ionic threshold prior to protein elution and thus allowing elution of active proteins in very small volumes of liquid. The invention also includes method for extracting biomolecules using the columns described herein.

### BRIEF DESCRIPTION OF THE FIGURES

FIG. 1 depicts an embodiment of the invention where the extraction column can take the form of a pipette tip.

FIG. 2 depicts a solid phase extraction using a conventional affinity resin.

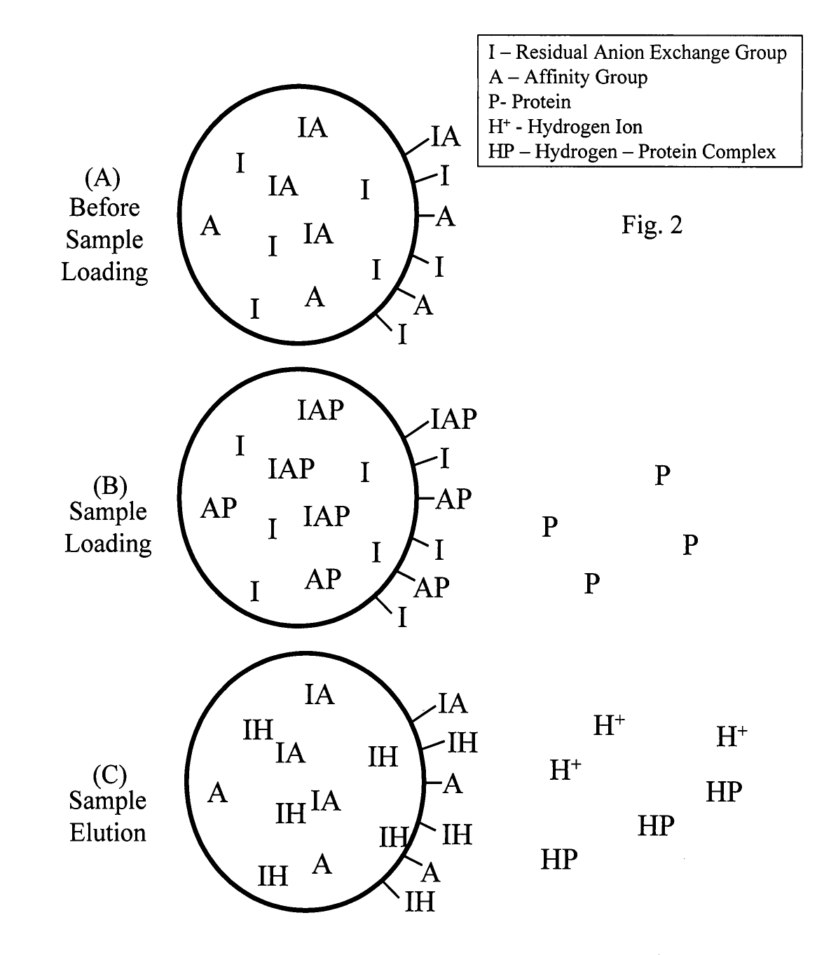

FIG. 3 depicts an embodiment of non-porous resin of the instant invention.

FIG. 4 depicts an embodiment of a porous resin of the instant invention.

FIG. 5 depicts a Carboxyl-reactive chemistry resin synthesis route.

### DESCRIPTION OF SPECIFIC EMBODIMENTS OF THE INVENTION

This invention relates to methods and devices for extracting an analyte from a sample solution. The analytes can include biomolecules, particularly biological macromolecules such as proteins and polypeptides. The device and method of this invention are particularly useful in proteomics for sample preparation and analysis with analytical technologies employing biochips, mass spectrometry and instrumentation and biological assays. The extraction process generally results in the enrichment, concentration, and/or purification of an analyte or analytes of interest.

In U.S. patent application Ser. No. 10/620,155, incorporated by reference herein in its entirety, methods and devices for performing low dead column extractions are described. The instant specification, inter alia, expands upon the concepts described in that application.

Before describing the present invention in detail, it is to be understood that this invention is not limited to specific embodiments described herein. It is also to be understood that the terminology used herein for the purpose of describing particular embodiments is not intended to be limiting. As used in this specification and the appended claims, the singular forms “a”, “an” and “the” include plural referents unless the context clearly dictates otherwise. Thus, for example, reference to polymer bearing a protected carbonyl would include a polymer bearing two or more protected carbonyls, and the like.

Although any methods and materials similar or equivalent to those described herein can be used in the practice or testing of the present invention, specific examples of appropriate materials and methods are described herein.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### DEFINITIONS

In describing and claiming the present invention, the following terminology will be used in accordance with the definitions set out below.

“Analyte” refers to a component of a sample which is desirably retained and detected. The term can refer to a single component or a set of components in the sample.

Adsorb” refers to the detectable binding between binding functionalities of an adsorbent (e.g., a hydrogel material or uniform particles) and an analyte either before or after washing with an eluant (selectivity threshold modifier).

The terms “dispense” and “expel” are used synonymously herein.

“Active proteins” are defined as proteins that are functional, active or in their native state. The terms “active proteins” and “native proteins” are used interchangeably herein.

The term “bed volume” as used herein is defined as the volume of a packed bed of extraction medium in an extraction column. Depending on how densely the bed is packed, the volume of the extraction medium in the column bed is typically about one third to two thirds of the total bed volume. The remaining volume not occupied by the extraction medium is the interstitial volume.; well-packed beds have less space between the beads and hence generally have lower interstitial volumes.

The term “interstitial volume” of the bed refers to the volume of the bed of extraction medium that is accessible to solvent, e.g., aqueous sample solutions, wash solutions and desorption solvents. For example, in the case where the extraction medium is a chromatography bead (e.g., agarose or sepharose), the interstitial volume of the bed constitutes the solvent accessible volume between the beads, as well as any solvent accessible internal regions of the bead, e.g., solvent accessible pores. The interstitial volume of the bed represents the minimum volume of liquid required to saturate the column bed.

The term “dead volume” as used herein with respect to a column is defined as the interstitial volume of the extraction bed, tubes, membrane or frits, and passageways in a column. Some preferred embodiments of the invention involve the use of low dead volume columns, as described in more detail in U.S. patent application Ser. No. 10/620,155.

The term “elution volume” as used herein is defined as the volume of desorption or elution liquid into which the analytes are desorbed and collected. The terms “desorption solvent,” “elution liquid” and the like are used interchangeably herein.

The term “enrichment factor” as used herein is defined as the ratio of the sample volume divided by the elution volume, assuming that there is no contribution of liquid coming from the dead volume. To the extent that the dead volume either dilutes the analytes or prevents complete adsorption, the enrichment factor is reduced.

The terms “extraction column” and “extraction tip” as used herein are defined as a column device used in combination with a pump, the column device containing a packed bed of solid phase extraction material, i.e., extraction media.

The term “frit” as used herein is defined as porous material for holding the extraction medium in place in a column. An extraction media chamber is typically defined by a top and bottom frit positioned in an extraction column. In some embodiments of the invention the frit is a thin, low pore volume filter, e.g., a membrane screen.

The term “lower column body” as used herein is defined as the column bed and bottom membrane screen of a column.

The term “membrane screen” as used herein is defined as a woven or non-woven fabric or screen for holding the column packing in place in the column bed, the membranes having a low dead volume. The membranes are of sufficient strength to withstand packing and use of the column bed and of sufficient porosity to allow passage of liquids through the column bed. The membrane is thin enough so that it can be sealed around the perimeter or circumference of the membrane screen so that the liquids flow through the screen.

The term “sample volume”, as used herein is defined as the volume of the liquid of the original sample solution from which the analytes are separated or purified.

The term “upper column body”, as used herein is defined as the chamber and top membrane screen of a column.

The term “biomolecule” as used herein refers to biomolecule derived from a biological system. The term includes biological macromolecules, such as a proteins, peptides, and nucleic acids.

The term “spacer arm” refers to the group to which the affinity or ion exchange moiety is bound.

### Extraction Columns

In accordance with the present invention there may be employed conventional chemistry, biological and analytical techniques within the skill of the art. Such techniques are explained fully in the literature. See, e.g. Chromatography, 5^{th} edition, PART A: FUNDAMENTALS AND TECHNIQUES, editor: E. Heftmann, Elsevier Science Publishing Company, New York (1992); ADVANCED CHROMATOGRAPHIC AND ELECTROMIGRATION METHODS IN BIOSCIENCES, editor: Z. Deyl, Elsevier Science BV, Amsterdam, The Netherlands, (1998); CHROMATOGRAPHY TODAY, Colin F. Poole and Salwa K. Poole, and Elsevier Science Publishing Company, New York, (1991).

In some embodiments of the subject invention the packed bed of extraction medium is contained in a column, e.g., a low dead volume column. Non-limiting examples of suitable columns, particularly low dead volume columns, are presented herein. It is to be understood that the subject invention is not to be construed as limited to the use of extraction beds in low dead volume columns, or in columns in general. For example, the invention is equally applicable to use with a packed bed of extraction medium as a component of a multi-well plate.

### Column Body

The column body is a tube having two open ends connected by an open channel, sometimes referred to as a through passageway. The tube can be in any shape, including but not limited to cylindrical or frustoconical, and of any dimensions consistent with the function of the column as described herein. In certain embodiments of the invention the column body takes the form of a pipette tip, a syringe, a luer adapter or similar tubular bodies. In embodiments where the column body is a pipette tip, the end of the tip wherein the bed of extraction medium is placed can take any of a number of geometries, e.g., it can be tapered or cylindrical. In some case a cylindrical channel of relatively constant radius can be preferable to a tapered tip, for a variety of reasons, e.g., solution flows through the bed at a uniform rate, rather than varying as a function of a variable channel diameter.

In some embodiments, one of the open ends of the column, sometimes referred to herein as the open upper end of the column, is adapted for attachment to a pump, either directly or indirectly. In some embodiments of the invention the upper open end is operatively attached to a pump, whereby the pump can be used for aspirating (i.e., drawing) a fluid into the extraction column through the open lower end of the column, and optionally for discharging (i.e., expelling) fluid out through the open lower end of the column. Thus, it is a feature certain embodiments of the present invention that fluid enters and exits the extraction column through the same open end of the column, typically the open lower end. This is in contradistinction with the operation of some extraction columns, where fluid enters the column through one open end and exits through the other end after traveling through the extraction medium, i.e., similar to conventional column chromatography. The fluid can be a liquid, such as a sample solution, wash solution or desorption solvent. The fluid can also be a gas, e.g., air used to blow liquid out of the extraction column.

In other embodiments of the present invention, fluid enters the column through one end and exits through the other. In some embodiments, the invention provides extraction methods that involve a hybrid approach; that is, one or more fluids enter the column through one end and exit through the other, and one more fluids enter and exit the column through the same open end of the column, e.g., the lower end. Thus, for example, in some methods the sample solution and/or wash solution are introduced through the top of the column and exit through the bottom end, while the desorption solution enters and exits through the bottom opening of the column. Aspiration and discharge of solution through the same end of the column can be particularly advantageous in procedures designed to minimize sample loss, particularly when small volumes of liquid are used. A good example would be a procedure that employs a very small volume of desorption solvent, e.g., a procedure involving a high enrichment factor.

The column body can be can be composed of any material that is sufficiently non-porous that it can retain fluid and that is compatible with the solutions, media, pumps and analytes used. A material should be employed that does not substantially react with substances it will contact during use of the extraction column, e.g., the sample solutions, the analyte of interest, the extraction medium and desorption solvent. A wide range of suitable materials are available and known to one of skill in the art, and the choice is one of design. Various plastics make ideal column body materials, but other materials such as glass, ceramics or metals could be used in some embodiments of the invention. Some examples of preferred materials include polysulfone, polypropylene, polyethylene, polyethyleneterephthalate, polyethersulfone, polytetrafluoroethylene, cellulose acetate, cellulose acetate butyrate, acrylonitrile PVC copolymer, polystyrene, polystyrene/acrylonitrile copolymer, polyvinylidene fluoride, glass, metal, silica, and combinations of the above listed materials.

### Extraction Media

The extraction medium used in the column is preferably a form of water-insoluble particle (e.g., a porous or non-porous bead) that has an affinity for an analyte of interest. Typically the analyte of interest is a protein or peptide. In general, the term “extraction medium” is used in a broad sense to encompass any media capable of effecting separation, either partial or complete, of an analyte from another. In some embodiments, the extraction process is affinity and the medium is comprised of an affinity group having an affinity for the analyte. The terms “separation column” and “extraction column” can be used interchangeably. The term “analyte” can refer to any compound of interest, e.g. an active protein, to be retained, analyzed or simply removed from a solution.

The bed volume of the extraction medium used in the extraction columns of the invention is typically small, typically in the range of 0.1-1000 μL, preferably in the range of 0.1-100 μL, e.g., in a range having a lower limit of 0.1, 0.5, 1, 1.5, 2, 2.5, 3, 5 or 10 μL; and an upper limit of 5, 10, 15, 20, 30, 40 50, 60, 70, 80, 90, 100, 150, 200, 300, 400 or 500 μL. The low bed volume contributes to a low interstitial volume of the bed, reducing the dead volume of the column, thereby facilitating the recovery of analyte in a small volume of desorption solvent.

The low bed volumes employed in certain embodiments allow for the use of relatively small amounts of extraction media, e.g., soft, gel-type beads. For example, some embodiments of the invention employ a bed of extraction medium having a dry weight of less than 1 gram (e.g., in the range of 0.001-1 g, 0.005-1 g, 0.01-1 g or 0.02-1 g), less than 100 mg (e.g., in the range of 0.1-100 mg, 0.5-100 mg, 1-100 mg 2-100 mg, or 10-100 mg), less than 10 mg (e.g., in the range of 0.1-10 mg, 0.5-10 mg, 1-10 mg or 2-10 mg), less than 2 mg (e.g., in the range of 0.1-2 mg, 0.5-2 mg or 1-2 mg), or less than 1 mg (e.g., in the range of 0.1-1 mg or 0.5-1 mg).

Many of the extraction media suitable for use in the invention are selected from a variety of classes of chromatography media. It has been found that many of these chromatography media and the associated chemistries are suited for use as solid phase extraction media in the devices and methods of this invention.

Thus, examples of suitable extraction media include resin beads used for extraction and/or chromatography. Examples of suitable resins include gel resins, pellicular resins, and macroporous resins.

The term “gel resin” refers to a resin comprising low-crosslinked bead materials that can swell in a solvent, e.g., upon hydration. Crosslinking refers to the physical linking of the polymer chains that form the beads. The physical linking is normally accomplished through a crosslinking monomer that contains bi-polymerizing functionality so that during the polymerization process, the molecule can be incorporated into two different polymer chains. The degree of crosslinking for a particular material can range from 0.1 to 30%, with 0.5 to 10% normally used. 1 to 5% crosslinking is most common. A lower degree of crosslinking renders the bead more permeable to solvent, thus making the functional sites within the bead more accessible to analyte. However, a low crosslinked bead can be deformed easily, and should only be used if the flow of eluent through the bed is slow enough or gentle enough to prevent closing the interstitial spaces between the beads, which could then lead to catastrophic collapse of the bed. Higher crosslinked materials swell less and may prevent access of the analytes and desorption materials to the interior functional groups within the bead. Generally, it is desirable to use as low a level of crosslinking as possible, so long is it is sufficient to withstand collapse of the bed. This means that in conventional gel-packed columns, slow flow rates may have to be used. In the present invention the back pressure is very low, and high liquid flow rates can be used without collapsing the bed. Surprisingly, using these high solvent velocities does not appear to reduce the capacity or usefulness of the bead materials. Common gel resins include agarose, sepharose, polystyrene, polyacrylate, cellulose and other substrates. Gel resins can be non-porous or micro-porous beads.

The term “pellicular resins” refers to materials in which the functional groups are on the surface of the bead or in a thin layer on the surface of the bead. The interior of the bead is solid, usually highly crosslinked, and usually inaccessible to the solvent and analytes. Pellicular resins generally have lower capacities than gel and macroporous resins.

The term “macroporous resin” refers to highly crosslinked resins having high surface area due to a physical porous structure that formed during the polymerization process. Generally an inert material (such as a solid or a liquid that does not solvate the polymer that is formed) is polymerized with the bead and then later washed out, leaving a porous structure. Crosslinking of macroporous materials range from 5% to 90% with perhaps a 25 to 55% crosslinking the most common materials. Macroporous resins behave similar to pellicular resins except that in effect much more surface area is available for interaction of analyte with resin functional groups.

Examples of resins beads include polystyrene/divinylbenzene copolymers, poly methylmethacrylate, protein G beads (e.g., for IgG protein purification), MEP Hypercel™ beads (e.g., for IgG protein purification), affinity phase beads (e.g., for protein purification), ion exchange phase beads (e.g., for protein or nucleic acid purification), hydrophobic interaction beads (e.g., for protein purification), reverse phase beads (e.g., for nucleic acid or protein purification), and beads having an affinity for molecules analyzed by label-free detection. Silica beads including surface-reacted silica and controlled pore glass beads are also suitable.

Soft gel resin beads, such as agarose and sepharose based beads, are found to work surprisingly well in columns and methods of this invention. Thus, in some embodiments of the instant invention, the gel resin is comprised of agarose or sepharose. In certain embodiments, the gel resin beads are further comprised of affinity groups having an affinity for the protein analyte.

In conventional chromatography fast flow rates can result in bead compression, which results in increased back pressure and adversely impacts the ability to use these gels with faster flow rates. In the present invention relatively small bed volumes can be used, which allows for the use of high flow rates with a minimal amount of bead compression.

### Resin Synthesis

Affinity resins and other resins used for the separation or extraction of biomolecules can contain ion exchange groups that are left over from the resin synthesis and are not active in the separation of a biomolecule. Care must be taken to avoid resins that possess these residual ion exchange groups when selecting affinity resins or when derivatizing resins with an affinity group. For example, Protein A affinity resin may contain ion exchange sites that are left over from the synthesis of the resin. The attachment of the protein A molecule to the agarose substrate is performed through an ion exchange site. Not all of the ion exchange sites are completely reacted in the synthesis process and some remain present after the synthesis is complete. Or the synthesis procedure incorporates an ion exchange group in the final product. The residual ion exchange groups can be strong acid groups such as sulfonic acid or phosphoric acid, weak acid groups such as carboxylic acid, strong base groups such as quaternary amine or weak base groups such as primary, secondary, or tertiary amines. Often, the residual ion exchange group is a weak base amine. In any case, these residual ion exchange groups may take up hydrogen cation or other cations if they are acidic or hydrogen, base or anions if they are basic. These ion exchange sites do not interfere with the capture of an antibody protein. But they do negatively impact the efficacy of the elution process as described above in the section entitled BACKGROUND OF THE INVENTION.

Thus, in some embodiments the invention comprises a resin that lacks residual ion exchange sites. The absent ion exchange site can be an anion (such as a carboxyl group) or an amine. In certain embodiments the invention comprises a solid phase extraction column filled with a resin that lacks residual ion exchange sites. In certain embodiments the resin that lacks residual ion exchange sites is a gel resin, such as agarose or sepharose. In some embodiments the column is a pipette tip column.

In other embodiments the invention is a column that does not require an ionic threshold to be reached before the biomolecule is eluted effectively. In certain embodiments the column is a pipette tip column.

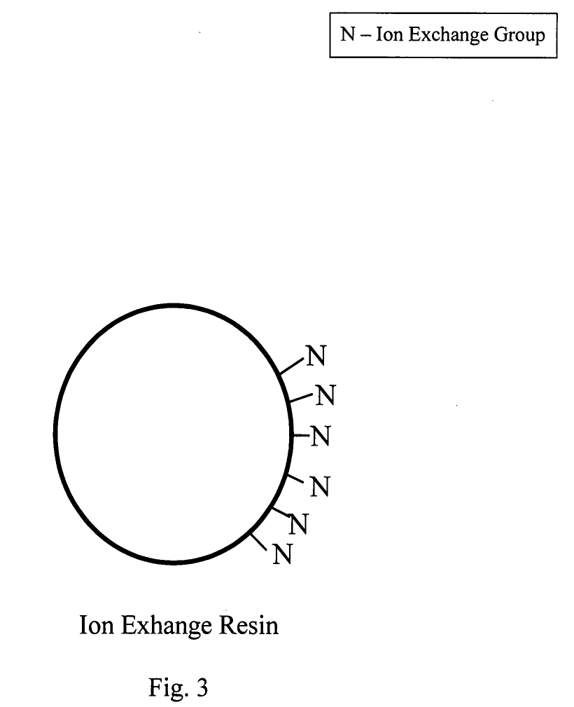

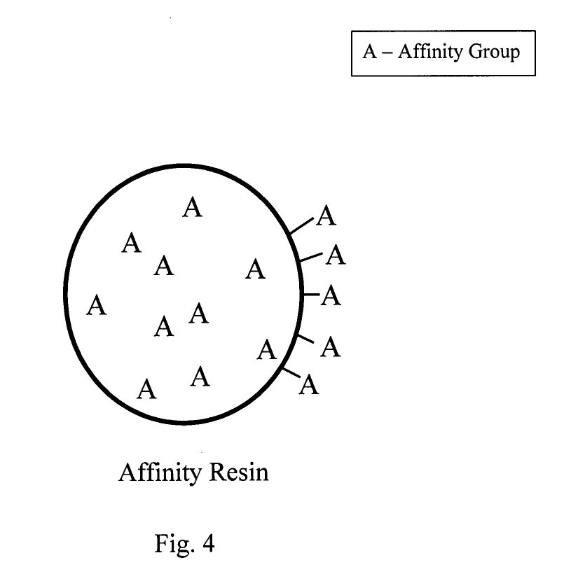

In some embodiments the resin is non-porous and contains no interior sites as depicted in FIG. 3. In other embodiments the resin is a porous resin, such as an affinity resin that contains no residual ion exchange sites as shown in FIG. 4.

Methods for synthesizing an affinity adsorbent are based on one of two general procedures: either the ligand or ligand analogue is reacted with adsorbent already possessing the spacer arm, or a ligand analogue containing the spacer arm is reacted with activated adsorbent. The spacer arm must have reactive ends for attachment to both matrix and ligand. In some cases the ligand-spacer arm combination is synthesized from smaller components rather than an attempt made to link them in one step.

The chemistry of the activation process involves the reaction of hydroxyl groups on the agarose matrix. One of the most common reactions has been cyanogen bromide. The original method has been superseded by a simpler technique in which the reaction is complete in a few minutes, Cyanogen-activated agarose available commercially has been widely used. The reactions of cyanogen bromide are complex; both cyclic and acyclic imidocarbonates are formed, with some carbamates and carbonates as reaction side products. But the principal reaction product is the cyanate ester. Also, the imidocarbonates, being unstable in acid, are destroyed by an acid treatment in the Pharmacia preparation of cyanogen-bromide-activated sepharose.

The activated agarose reacts swiftly in weakly alkaline conditions (pH 9-10) with primary amines to give principally the isourea derivative. Some simultaneous aqueous hydrolysis gives carbamates and carbonates.

The isourea substituent may be charged at neutral pH, which can influence the behavior of affinity adsorbents by introducing an element of anion exchange character. On the other hand, many of the ligands that are attached are negatively charged, so the isourea derivative may cancel out possible cation exchange effects, though leaving dipolar ion characteristics.

Although cyanogen bromide activation is still the most widely used method, especially for attachment of proteins through lysine or amine groups, other more convenient and efficient methods are now available and undoubtedly will eventually supplant the cyanogen bromide procedure. The first of these is “epoxy activation,” in which an epoxy group is introduced, using 1,4-butanediol diglycidyl ether (a bisoxirane).

In the latter case a spacer arm is automatically inserted, since there are 11 atoms between the matrix and the reactive epoxy group. Epoxy groups are somewhat more reactive than cyanates. They combine with primary amines to form ether linkages. The coupling is carried out at pH 8.5-11. The reaction with hydroxyls is slow, taking about 24 hours at room temperature.

The next method involves activation with 1,1′-carbonyldiimidazole, which gives a product just as reactive as the cyanate; coupling (again to a primary amine) does not leave a positive charge, but a urethane linkage. The reagent is also less noxious than either cyanogen bromide or bisoxiranes.

A more recent method, which may well be the best, involves the use of toluene sulfonyl chloride (tosyl chloride), or the more reactive 3,3,3-trifluoroethanesulfonyl chloride (tresyl chloride). The tosyl group introduced on activation reacts smoothly and rapidly to give the very stable secondary amine from the primary amine ligand. At neutral pH the secondary amine group may be charged, so the ultimate behavior may be similar to that of cyanogen bromide activation, but the linkage is much more stable.

Yet another chemistry involves the reaction of divinyl sulfone with the matrix to yield a vinyl-sulfone-activated form, which also reacts with amino groups and, more slowly with hydroxyls.

In practice, the amines in the examples above is usually either the ligand with spacer arm ending with the amine already attached, or hexamethylenediamine, which gives a spacer arm ending with another primary amine; i.e.

>*+NH₂(CH₂)6NH₂→>—NH(CH₂)₆NH₂

### The further chemistry of attachment of particular types of ligands and spacer arms is extensively reviewed elsewhere.

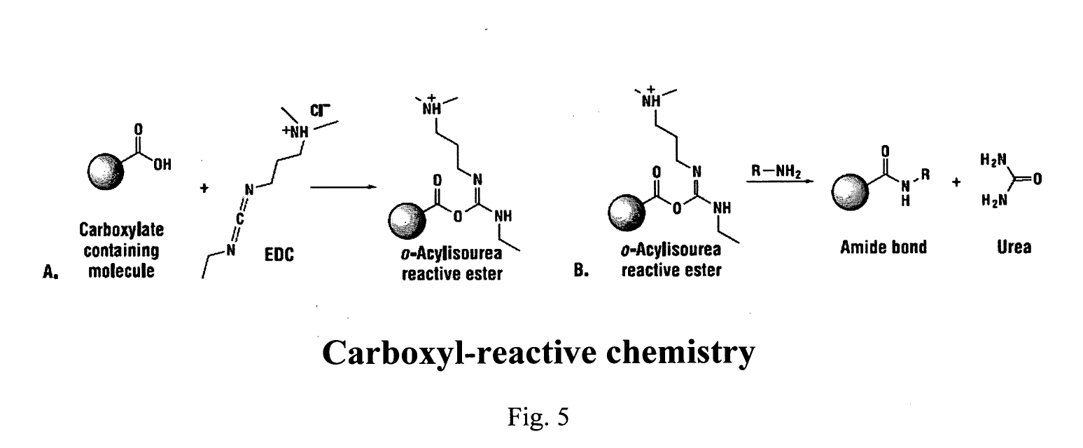

EDC (Product # 22980, 22981, Pierce, Inc.) reacts with carboxylic acid group and activates the carboxyl group to form an active O-acylisourea intermediate, allowing it to be coupled to the amino group in the reaction mixture. An EDC byproduct is released as a soluble urea derivative after displacement by the nucleophile (FIG. 5). The O-acylisourea intermediate is unstable in aqueous solutions, making it ineffective in two-step conjugation procedures without increasing the stability of the intermediate using N-hydroxysuccinimide. This intermediate reacts with a primary amine to form an amide derivative. There are no residual anion exchange sites. Failure to react with an amine results in hydrolysis of the intermediate, regeneration of the carboxyls and the release of an N-unsubstituted urea. The crosslinking reaction is usually performed between pH 4.5 to 5 and requires only a few minutes for many applications. However, the yield of the reaction is similar at pH from 4.5 to 7.5.

Another resin synthesis option involves end-capping ion exchange sites following resin derivatization. An example synthesis route involves carboxyl-reactive chemistry. Carbodiimides couple carboxyls to primary amines or hydrazides, resulting in the formation of amide or hydrazone bonds. Carbodiimides are unlike other conjugation reactions in that no spacer exists between the molecules being coupled.

Sources of carboxy resins include methacrylate resins from Tosoh BioScience: CM 650S (20-50 μm), CM 650M (40-90 μm), CM 650C (50-150 um), and Toyopearl AF-carboxy 650M (40-90 μm), CM MacroPrep Media from BioRad (50 μm) and carboxymethyl sepharose resins from GE (45-165 μm).

Thus, in some embodiments the invention is a pipette tip column containing resin in which ion exchange sites were eliminated by end-capping during the resin synthesis. In particular, end-capping may be accomplished using carboxyl-reactive chemistry. In some embodiments the invention is a pipette tip column containing agarose or sepharose in which the hydroxyl groups were activated using vinyl sulfone, followed by attachment of Protein A.

Polystyrene and hydroxyl polystyrene beads are available from Dow, Rohm and Haas, BioRad, PolyScience, Rapp Polymere and others. The surface of polystyrene substrates has been modified by wet chemistry consisting of a treatment with sodium hydroxide in a water-methanol solution at 50° C. for 15 h, under air atmosphere to produce a surface carboxylic acid. The alkyl group on polyethylstyrene in a polystyrene bead can surface oxidized to a carboxylic acid with potassium permanganate. Polystyrene beads with hydroxyl groups, primary alcohols, can be oxidized to carboxylic acids by many strong oxidizing agents include chromic acid, permanganate, and nitric acid. One example of a suitable resin is 106-125μ polystyrene beads from PolyScience, Inc.

Silane coupling agents are typically used in the surface treatment of silica gel or controlled pore glass (for reference, see Silane Coupling Agents. Connecting Across Boundaries www.gelest.com/company/pdfs/couplingagents.pdf, U.S. Pat. No. 5,374,755 and Silica-Based Packing Materials for PREP HPLC, SFC and SMB, ANALUSIS MAGAZINE, 1998, 26, No. 7, © EDP Sciences, Wiley-VCH). Example of suitable resins include SilicAR V54 silica gel 35-60 μm 22 Angstrom pores or 6447 silica gel 75-160 μm 60 Angstrom pores from Mallinckrodt, controlled-pore glass (Biosearch Tecnologies, Inc., Monomer Sciences Inc., SBS Genetech, Annovis Inc.) and others. Organofunctional silanes can undergo subsequent condensation with hydroxyl groups on the silica to yield a desired chemical reactivity or functionality. One common stationary phase is a silica which has been treated with RMe₂SiCl, where R is a straight chain alkyl group such as C₁₈H₃₇ or C₈H₁₇. Virtually any type of functional group can be attached to silica depending on the silane reagent used for attachment. The following Table shows some example sorbents and bonding chemistries. The use of these resins with the above-described synthesis routes can provide final products that lack residual ion exchange sites.

### Silane Coupling Chemistries

| Sorbent | Bonding Chemistry |
|---|---|
| C18 | X—Si(CH₃)₂C₁₈H₃₇ |
| C8 | X—Si(CH₃)₂C₈H₁₇ |
| C2 | X—SiC₂H₅ |
| Amino Propyl (NH2) | X—Si(CH₂)₃NH₂ |
| Cyano Propyl (CN) | X—Si(CH₃)(CH₂)₃CN |
| Diol | X—Si(CH₂)₃OCH₂CH(OH)CH₂OH |
| Carboxy | X—Si(CH₃)(CH₂)₃COOH |

where X is chloro or trimethoxy for example

The average particle diameters of beads of the invention are typically in the range of about 1 μm to several millimeters, e.g., diameters in ranges having lower limits of 1 μm, 5 μm, 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 10 μm, 20 μm, 30 μm, 40 μm, 50 μm, 60 μm, 70 μm, 80 μm, 90 μm, 100 μm, 150 μm, 200 μm, 300 μm, 500 μm, 750 μm, 1 mm, 2 mm, or 3 mm. In some embodiments of the invention the bead size is 200-400 mesh.

The bead size that may be used depends somewhat on the bed volume and the cross sectional area of the column. A lower bed volume column will tolerate a smaller bead size without generating the high backpressures that could burst a thin membrane frit. For example a bed volume of 0.1 to 1 μL bed, can tolerate 5 to 10 μm particles. Larger beds (up to about 50 μL) normally have beads sizes of 30-150 μm or higher. The upper range of particle size is dependant on the diameter of the column bed. The bead diameter size should not be more than 50% of the bed diameter, and preferably less than 10% of the bed diameter.

The extraction chemistry employed in the present invention can take any of a wide variety of forms. For example, the extraction medium can be selected from, or based on, any of the extraction chemistries used in solid-phase extraction and/or chromatography. Because the invention is particularly suited to the purification and/or concentration of biomolecules such as active proteins and polypeptides, extraction surfaces capable of adsorbing such molecules are particularly relevant. See, e.g., SEPARATION AND SCIENCE TECHNOLOGY Vol. 2.: HANDBOOK OF BIOSEPARATIONS, edited by Satinder Ahuja, Academic Press (2000).

Affinity extractions use a technique in which a bio-specific adsorbent is prepared by coupling a specific ligand (such as an enzyme, antigen, or hormone) for the analyte, (e.g., macromolecule) of interest to a solid support. This immobilized ligand will interact selectively with molecules that can bind to it. Molecules that will not bind elute un-retained. The interaction is selective and reversible. The references listed below show examples of the types of affinity groups that can be employed in the practice of this invention are hereby incorporated by reference herein in their entireties. Antibody Purification Handbook, Amersham Biosciences, Edition AB, 18-1037-46 (2002); Protein Purification Handbook, Amersham Biosciences, Edition AC, 18-1132-29 (2001); Affinity Chromatography Principles and Methods, Amersham Pharmacia Biotech, Edition AC, 18-1022-29 (2001); The Recombinant Protein Handbook, Amersham Pharmacia Biotech, Edition AB, 18-1142-75 (2002); and Protein Purification: Principles, High Resolution Methods, and Applications, Jan-Christen Janson (Editor), Lars G. Ryden (Editor), Wiley, John & Sons, Incorporated (1989).

Examples of suitable affinity binding agents are summarized in Table I, wherein the affinity agents are from one or more of the following interaction categories:
1. Chelating metal—ligand interaction
2. Protein—Protein interaction
3. Organic molecule or moiety—Protein interaction
4. Sugar—Protein interaction
5. Nucleic acid—Protein interaction

TABLE I

| Examples of Affinity | | |
|---|---|---|
| molecule or moiety fixed at | | Interaction |
| surface | Captured biomolecule | Category |
| Ni-NTA | His-tagged protein | 1 |
| Ni-NTA | His-tagged protein within a | 1, 2 |
| | multi-protein complex | |
| Fe-IDA | Phosphopeptides, | 1 |
| | phosphoproteins | |
| Fe-IDA | Phosphopeptides or | 1, 2 |
| | phosphoproteins within a | |
| | multi-protein complex | |
| Antibody or other Proteins | Protein antigen | 2 |
| Antibody or other Proteins | Small molecule-tagged | 3 |
| | protein | |
| Antibody or other Proteins | Small molecule-tagged | 2, 3 |
| | protein within a multi- | |
| | protein complex | |
| Antibody or other Proteins | Protein antigen within a | 2 |
| | multi-protein complex | |
| Antibody or other Proteins | Epitope-tagged protein | 2 |
| Antibody or other Proteins | Epitope-tagged protein | 2 |
| | within a multi-protein | |
| | complex | |
| Protein A, Protein G or | Antibody | 2 |
| Protein L | | |
| Protein A, Protein G or | Antibody | 2 |
| Protein L | | |
| ATP or ATP analogs; 5′- | Kinases, phosphatases | 3 |
| AMP | (proteins that requires ATP | |
| | for proper function) | |
| ATP or ATP analogs; 5′- | Kinase, phosphatases | 2, 3 |
| AMP | within multi-protein | |
| | complexes | |
| Cibacron 3G | Albumin | 3 |
| Heparin | DNA-binding protein | 4 |
| Heparin | DNA-binding proteins | 2, 4 |
| | within a multi-protein | |
| | complex | |
| Lectin | Glycopeptide or | 4 |
| | glycoprotein | |
| Lectin | Glycopeptide or | 2, 4 |
| | glycoprotein within a | |
| | multi-protein complex | |

In one aspect of the invention an extraction medium is used that contains a surface functionality that has an affinity for a protein fusion tag used for the purification of recombinant proteins. A wide variety of fusion tags and corresponding affinity groups are available and can be used in the practice of the invention.

U.S. patent application Ser. No. 10/620,155 describes in detail the use of specific affinity binding reagents in solid-phase extraction. Examples of specific affinity binding agents include proteins having an affinity for antibodies, Fc regions and/or Fab regions such as Protein G, Protein A, Protein A/G, and Protein L; chelated metals such as metal-NTA chelate (e.g., Nickel NTA, Copper NTA, Iron NTA, Cobalt NTA, Zinc NTA), metal-IDA chelate (e.g., Nickel IDA, Copper IDA, Iron IDA, Cobalt IDA) and metal-CMA (carboxymethylated aspartate) chelate (e.g., Nickel CMA, Copper CMA, Iron CMA, Cobalt CMA, Zinc CMA); glutathione surfaces—nucleotides, oligonucleotides, polynucleotides and their analogs (e.g., ATP); lectin surface—heparin surface—avidin or streptavidin surface, a peptide or peptide analog (e.g., that binds to a protease or other enzyme that acts upon polypeptides).

In some embodiments of the invention, the affinity binding reagent is one that recognizes one or more of the many affinity groups used as affinity tags in recombinant fusion proteins. Examples of such tags include poly-histidine tags (e.g., the 6×-His tag), which can be extracted using a chelated metal such as Ni-NTA-peptide sequences (such as the FLAG epitope) that are recognized by an immobilized antibody; biotin, which can be extracted using immobilized avidin or streptavidin; “calmodulin binding peptide” (or, CBP), recognized by calmodulin charged with calcium-glutathione S-transferase protein (GST), recognized by immobilized glutathione; maltose binding protein (MBP), recognized by amylose; the cellulose-binding domain tag, recognized by immobilized cellulose; a peptide with specific affinity for S-protein (derived from ribonuclease A); and the peptide sequence tag CCxxCC (where xx is any amino acid, such as RE), which binds to the affinity binding agent bis-arsenical fluorescein (FlAsH dye).

Antibodies can be extracted using, for example, proteins such as protein A, protein G, protein L, hybrids of these, or by other antibodies (e.g., an anti-IgE for purifying IgE).

Chelated metals are not only useful for purifying poly-his tagged proteins, but also other non-tagged proteins that have an intrinsic affinity for the chelated metal, e.g., phosphopeptides and phosphoproteins.

Antibodies can also be useful for purifying non-tagged proteins to which they have an affinity, e.g., by using antibodies with affinity for a specific phosphorylation site or phosphorylated amino acids.

### Frits

In some embodiments of the invention one or more frits is used to contain the bed of extraction medium in, for example, a column. Frits can take a variety of forms, and can be constructed from a variety of materials, e.g., glass, ceramic, metal, fiber. Some embodiments of the invention employ frits having a low pore volume, which contribute to reducing dead volume. The frits of the invention are porous, since it is necessary for fluid to be able to pass through the frit. The frit should have sufficient structural strength so that frit integrity can contain the extraction medium in the column. It is desirable that the frit have little or no affinity for chemicals with which it will come into contact during the extraction process, particularly the analyte of interest. In many embodiments of the invention the analyte of interest is a biomolecule, particularly a biological macromolecule. Thus in many embodiments of the invention it desirable to use a frit that has a minimal tendency to bind or otherwise interact with biological macromolecules, particularly proteins, peptides and nucleic acids.

Frits of various pores sizes and pore densities may be used provided the free flow of liquid is possible and the beads are held in place within the extraction medium bed.

In one embodiment, one frit (e.g., a lower frit) is bonded to and extends across the open channel of the column body. A second frit is bonded to and extends across the open channel between the bottom frit and the open upper end of the column body.

In this embodiment, the top frit, bottom frit and column body (i.e., the inner surface of the channel) define an extraction media chamber wherein a bed of extraction medium is positioned. The frits should be securely attached to the column body and extend across the opening and/or open end so as to completely occlude the channel, thereby substantially confining the bed of extraction medium inside the extraction media chamber. In some embodiments of the invention the bed of extraction medium occupies at least 80% of the volume of the extraction media chamber, more preferably 90%, 95%, 99%, or substantially 100% of the volume. In some embodiments of the invention the space between the extraction medium bed and the upper and lower frits is negligible, i.e., the frits are in substantial contact with upper and lower surfaces of the extraction medium bed, holding a well-packed bed of extraction medium securely in place.

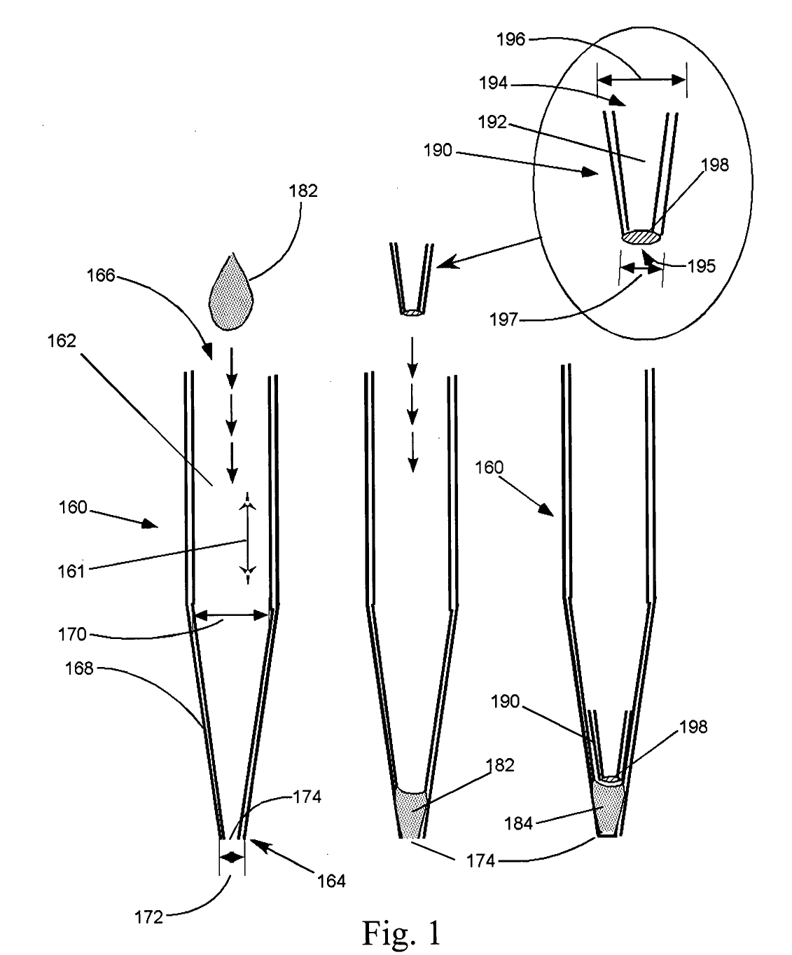

In some embodiments of the invention the bottom frit is located at the open lower end of the column body. This configuration is shown in FIG. 1 but is not required, i.e., in some embodiments the bottom frit is located at some distance up the column body from the open lower end. However, in view of the advantage that comes with minimizing dead volume in the column, it is desirable that the lower frit and extraction media chamber be located at or near the lower end. In some cases this can mean that the bottom frit is attached to the face of the open lower end, as shown in FIG. 1. However, in some cases there can be some portion of the lower end extending beyond the bottom frit. For the purposes of this invention, so long as the length of this extension is such that it does not substantially introduce dead volume into the extraction column or otherwise adversely impact the function of the column, the bottom frit is considered to be located at the lower end of the column body. In some embodiments of the invention the volume defined by the bottom frit, channel surface, and the face of the open lower end (i.e., the channel volume below the bottom frit) is less than the volume of the extraction media chamber, or less than 10% of the volume of the extraction media chamber, or less than 1% of the volume of the extraction media chamber.

In some embodiments of the invention, the extraction media chamber is positioned near one end of the column, which for purposes of explanation will be described as the bottom end of the column. The area of the column body channel above the extraction media chamber can be quite large in relation to the size of the extraction media chamber. For example, in some embodiments the volume of the extraction chamber is equal to less than 50%, less than 20, less than 10%, less than 5%, less than 2%, less than 1% or less than 0.5% of the total internal volume of the column body. In operation, solvent can flow through the open lower end of the column, through the bed of extraction medium and out of the extraction media chamber into the section of the channel above the chamber. For example, when the column body is a pipette tip, the open upper end can be fitted to a pipettor and a solution drawn through the extraction medium and into the upper part of the channel.

The frits used in the invention are preferably characterized by having a low pore volume. Some embodiments of the invention employ frits having pore volumes of less than 1 microliter (e.g., in the range of 0.015-1 microliter, 0.03-1 microliter, 0.1-1 microliter or 0.5-1 microliter), preferably less than 0.5 microliter (e.g., in the range of 0.015-0.5 microliter, 0.03-0.5 microliter or 0.1-0.5 microliter), less than 0.1 microliter (e.g., in the range of 0.015-0.1 microliter or 0.03-0.1 microliter) or less than 0.03 microliters (e.g., in the range of 0.015-0.03 microliter).

Frits of the invention preferably have pore openings or mesh openings of a size in the range of about 5-100 μm, more preferably 10-100 μm, and still more preferably 15-50 μm, e.g., about 43 μm. The performance of the column is typically enhanced by the use of frits having pore or mesh openings sufficiently large so as to minimize the resistance to flow. The use of membrane screens as described herein typically provide this low resistance to flow and hence better flow rates, reduced back pressure and minimal distortion of the bed of extraction medium. The pore or mesh openings of course should not be so large that they are unable to adequately contain the extraction medium in the chamber.

Some frits used in the practice of the invention are characterized by having a low pore volume relative to the interstitial volume of the bed of extraction medium contained by the frit. Thus, in certain embodiments of the invention the frit pore volume is equal to 10% or less of the interstitial volume of the bed of extraction medium (e.g., in the range 0.1-10%, 0.25-10%, 1-10% or 5-10% of the interstitial volume), more preferably 5% or less of the interstitial volume of the bed of extraction medium (e.g., in the range 0.1-5%, 0.25-5% or 1-5% of the interstitial volume), and still more preferably 1% or less of the interstitial volume of the bed of extraction medium (e.g., in the range 0.01-1%, 0.05-1% or 0.1-1% of the interstitial volume).

The pore density will allow flow of the liquid through the membrane and is preferably 10% and higher to increase the flow rate that is possible and to reduce the time needed to process the sample.

Some embodiments of the invention employ a thin frit, preferably less than 350 μm in thickness (e.g., in the range of 20-350 μm, 40-350 μm, or 50-350 μm), more preferably less than 200 μm in thickness (e.g., in the range of 20-200 μm, 40-200 μm, or 50-200 μm), more preferably less than 100 μm in thickness (e.g., in the range of 20-100 μm, 40-100 μm, or 50-100 μm), and most preferably less than 75 μm in thickness (e.g., in the range of 20-75 μm, 40-75 μm, or 50-75 μm).

Some embodiments of the invention employ a membrane screen as the frit. The membrane screen should be strong enough to not only contain the extraction medium in the column bed, but also to avoid becoming detached or punctured during the actual packing of the medium into the column bed. Membranes can be fragile, and in some embodiments must be contained in a framework to maintain their integrity during use. However, it is desirable to use a membrane of sufficient strength such that it can be used without reliance on such a framework. The membrane screen should also be flexible so that it can conform to the column bed. This flexibility is advantageous in the packing process as it allows the membrane screen to conform to the bed of extraction medium, resulting in a reduction in dead volume.

The membrane can be a woven or non-woven mesh of fibers that may be a mesh weave, a random orientated mat of fibers i.e. a “polymer paper,” a spun bonded mesh, an etched or “pore drilled” paper or membrane such as nuclear track etched membrane or an electrolytic mesh (see, e.g., 5,556,598). The membrane may be, e.g., polymer, glass, or metal provided the membrane is low dead volume, allows movement of the various sample and processing liquids through the column bed, may be attached to the column body, is strong enough to withstand the bed packing process, is strong enough to hold the column bed of beads, and does not interfere with the extraction process i.e. does not adsorb or denature the sample molecules.

The frit can be attached to the column body by any means which results in a stable attachment. For example, the screen can be bonded to the column body through welding or gluing. Gluing can be done with any suitable glue, e.g., silicone, cyanoacrylate glue, epoxy glue, and the like. The glue or weld joint must have the strength required to withstand the process of packing the bed of extraction medium and to contain the extraction medium with the chamber. For glue joints, a glue should be selected employed that does not adsorb or denature the sample molecules.

For example, glue can be used to attach a membrane to the tip of a pipette tip-based extraction column, i.e., a column wherein the column body is a pipette tip. A suitable glue is applied to the end of the tip. In some cases, a rod may be inserted into the tip to prevent the glue from spreading beyond the face of the body. After the glue is applied, the tip is brought into contact with the membrane frit, thereby attaching the membrane to the tip. After attachment, the tip and membrane may be brought down against a hard flat surface and rubbed in a circular motion to ensure complete attachment of the membrane to the column body. After drying, the excess membrane may be trimmed from the column with a razor blade.

Alternatively, the column body can be welded to the membrane by melting the body into the membrane, or melting the membrane into the body, or both. In one method, a membrane is chosen such that its melting temperature is higher than the melting temperature of the body. The membrane is placed on a surface, and the body is brought down to the membrane and heated, whereby the face of the body will melt and weld the membrane to the body. The body may be heated by any of a variety of means, e.g., with a hot flat surface, hot air or ultrasonically. Immediately after welding, the weld may be cooled with air or other gas to improve the likelihood that the weld does not break apart.

Alternatively, a frit can be attached by means of an annular pip, as described in U.S. Pat. No. 5,833,927. This mode of attachment is particularly suited to embodiment where the frit is a membrane screen.

The frits of the invention, e.g., a membrane screen, can be made from any material that has the required physical properties as described herein. Examples of suitable materials include nylon, polyester, polyamide, polycarbonate, cellulose, polyethylene, nitrocellulose, cellulose acetate, polyvinylidine difluoride, polytetrafluoroethylene (PTFE), polypropylene, polysulfone, metal and glass. A specific example of a membrane screen is the 43 μm pore size Spectra/Mesh® polyester mesh material which is available from Spectrum Labs (Ranch Dominguez, Calif., PN 145837).

Pore size characteristics of membrane filters can be determined, for example, by use of method #F316-30, published by ASTM International, entitled “Standard Test Methods for Pore Size Characteristics of Membrane Filters by Bubble Point and Mean Flow Pore Test.”

The polarity of the membrane screen can be important. A hydrophilic screen will promote contact with the bed and promote the air-liquid interface setting up a surface tension. A hydrophobic screen would not promote this surface tension and therefore the threshold pressures to flow would be different. A hydrophilic screen is utilized in certain embodiments of the invention.

### Extraction Column Assembly

The extraction columns of the invention can be constructed by a variety of methods using the teaching supplied herein. In some embodiments the extraction column can be constructed by the engagement (i.e., attachment) of upper and lower tubular members (i.e., column bodies) that combine to form the extraction column. In certain embodiments of the column manufacturing process, the inner column body is stably affixed to the outer column body by frictional engagement with the surface of the open channel.

In some embodiments of this general method of column manufacture, the entire inner column body is disposed within the first open channel. In these embodiments the first open upper end is normally adapted for operable attachment to a pump, e.g., the outer column body is a pipette tip and the pump is a pipettor. In some embodiments, the outer diameter of the inner column body tapers towards its open lower end, and the open channel of the outer column body is tapered in the region where the inner column body frictionally engages the open channel, the tapers of the inner column body and open channel being complementary to one another. This complementarity of taper permits the two bodies to fit snuggly together and form a sealing attachment, such that the resulting column comprises a single open channel containing the bed of extraction medium bounded by the two frits.

FIG. 1 illustrates the construction of an example of this embodiment of the extraction columns of the invention. This example includes an outer column body 160 having a longitudinal axis 161, a central through passageway 162 (i.e., an open channel), an open lower end 164 for the uptake and/or expulsion of fluid, and an open upper end 166 for operable attachment to a pump, e.g., the open upper end is in communication with a pipettor or multi-channel pipettor. The communication can be direct or indirect, e.g., through one or more fittings, couplings or the like, so long as operation of the pump effects the pressure in the central through passageway 162. The outer column body includes a frustoconical section 168 of the through passageway 162, which is adjacent to the open lower end 164. The inner diameter of the frustoconical section decreases from a first inner diameter 170, at a position in the frustoconical section distal to the open lower end, to a second inner diameter 172 at the open lower end. A lower frit 174, preferably a membrane screen, is bonded to and extends across the open lower end 164. In one embodiment a membrane frit can be bound to the outer column body by methods described herein, such as by gluing or welding. To construct the column, a desired quantity of extraction medium 182, preferably in the form of a slurry, is introduced into the through passageway through the open upper end and positioned in the frustoconical section adjacent to the open lower end. The extraction medium preferably forms a packed bed in contact with the lower frit 174. The upper frit 198, lower frit 174, and the surface of the through passageway bounded by the upper and lower frits define an extraction media chamber 184. The amount of extraction medium introduced into the column is normally selected such that the resulting packed bed substantially fills the extraction media chamber, preferably making contact with the upper and lower frits.

The outer diameter of the frustoconical member decreases from a first outer diameter 196 at the open upper end to a second outer diameter 197 at the open lower end. The second outer diameter 197 is greater than the second inner diameter 172 and less than the first inner diameter 170. The first outer diameter 196 is less than or substantially equal to the first inner diameter 170. An upper frit 198 is bonded to and extends across the open lower end 195. The frustoconical member 190 is introduced into the through passageway of an outer column body containing a bed of extraction medium positioned at the lower frit 174. The tapered outer surface of the frustoconical member matches and the taper of the frustoconical section of the open passageway, and the two surfaces make a sealing contact. The extended frustoconical configuration of this embodiment of the ring facilitates the proper alignment and seating of the ring in the outer passageway.

Because of the friction fitting of frustoconical member 190 to the surface of the central through passageway, it is normally not necessary to use additional means to bond the upper frit to the column. If desired, one could use additional means of attachment, e.g., by bonding, gluing, welding, etc. In some embodiments, the inner surface of the frustoconical section is modified to improve the connection between the two elements, e.g., by including grooves, locking mechanisms, etc.

In the foregoing embodiments, the latitudinal cross sections of the frustoconical section are illustrated as circular in geometry. Alternatively, other geometries could be employed, e.g., oval, polygonal or otherwise. Whatever the geometries, the ring and frustoconical shapes should match to the extent required to achieve and adequately sealing engagement. The frits are preferably, but not necessarily, positioned in a parallel orientation with respect to one another and perpendicular to the longitudinal axis.

### Solvents

Extractions of the invention typically involve the loading of analyte in a sample solution, an optional wash with a rinse solution, and elution of the analyte into a desorption solution. The nature of these solutions will now be described in greater detail.

With regard to the sample solution, it typically consists of the analyte dissolved in a solvent in which the analyte is soluble, and in which the analyte will bind to the extraction surface. Preferably, the binding is strong, resulting in the binding of a substantial portion of the analyte, and optimally substantially all of the analyte will be bound under the loading protocol used in the procedure. The solvent should also be gentle, so that the native structure and function of the analyte is retained upon desorption from the extraction surface. Typically, in the case where the analyte is a biomolecule, the solvent is an aqueous solution, typically containing a buffer, salt, and/or surfactants to solubilize and stabilize the biomolecule. Examples of sample solutions include cells lysates, hybridoma growth medium, cell-free translation or transcription reaction mixtures, extracts from tissues, organs, or biological samples, and extracts derived from biological fluids.

It is important that the sample solvent not only solubilize the analyte, but also that it is compatible with binding to the extraction phase. For example, where the extraction phase is based on affinity, the sample solution should be buffered to an appropriate pH, so the charge of the analyte is opposite that of the immobilized ion, and the ionic strength should promote efficient capture.

In some embodiments, a wash solution is passed through the extraction column between the sample solution and the desorption solution. In other embodiments, the wash is omitted. A wash solution, if used, should be selected such that it will remove non-desired contaminants with minimal loss or damage to the bound analyte. The properties of the wash solution are typically intermediate between that of the sample and desorption solutions.

In some cases more than one wash is performed. For example, capture of an antibody on a Protein A resin column is usually performed at pH 7.4. After capture, the beads are washed with a buffer of the same pH (7.4) to remove non-specifically bound material. Next, the column is washed with saline to remove the buffering capacity of any remaining wash solution. The saline wash removes the buffering capacity remaining from the first wash solution. This can be important when the captured analyte is eluted with the low pH desorption solution.

After the wash step, a desorption solution is passed through the column to elute the analyte. The terms “desorption” and “elution” are used interchangeably herein. The desorption solvent should be just strong enough to quantitatively desorb the analyte while leaving strongly bound interfering materials behind. The solvents are chosen to be compatible with the analyte and the ultimate detection method. In certain embodiments, the desorption solution has a low pH. In these embodiments, the desorption solution can be comprised of hydrogen cations that dissociate the protein analyte from the affinity group on the extraction medium.

Desorption solvent or solution can be introduced in a small volume. Another solvent can follow the desorption solution so that when the sample is transferred to a vial, a buffer is also deposited to give the sample the proper pH. An example of this is desorption from a protein G surface of IgG antibody which has been extracted from a hybridoma solution. In this example, 10 mM phosphoric acid at pH 2.5 is used to desorb the IgG from the pipette tip column. A 100 mM phosphate buffer plug at pH 7.5 follows the desorption solvent to bring the deposited solution to neutral pH. The deposited material can then be analyzed, e.g., by deposition on an SPR chip or any other analytical method.

In the case where the extraction involves binding of analyte to a specific cognate ligand molecule, e.g., an immobilized metal, the desorption solvent can contain a molecule that will interfere with such binding, e.g., imidazole or a metal chelator in the case of the immobilized metal.

Examples of suitable phases for solid phase extraction and desorption solvents are shown in Table A.

TABLE A

| Desorption | Affinity Phase |
|---|---|
| Solvent Features | Extraction |
| Typical solvent | High |
| polarity range | |
| Typical sample | H₂O, buffers |
| loading solvent | |
| Typical desorption | H₂O, buffers, pH, |
| solvent | competing reagents, |
| | heat, solvent polarity |
| Sample elution | Non-binding, low- |
| selectivity | binding, high-binding |
| Solvent change | Change pH, add |
| required to desorb | competing reagent, |
| | change solvent |
| | polarity, increase heat |

### Methods for Using the Extraction Columns

Generally the first step in an extraction procedure of the invention will involve introducing a sample solution containing an analyte of interest into a packed bed of extraction medium, typically in the form of a column as described above. The sample can be conveniently introduced into the separation bed by pumping the solution through the column. Note that the volume of sample solution can be much larger than the bed volume. The sample solution can optionally be passed through the column more than one time, e.g., by being pumped back and forth through the bed using repeated aspirate/expel cycles. This can improve adsorption of analyte, which can be particularly in cases where the analyte is of low abundance and hence maximum sample recovery is desired.

Certain embodiments of the invention are particularly suited to the processing of biological samples, where the analyte of interest is a biomolecule. Of particular relevance are biological macromolecules such as proteins, and polypeptides, or large complexes containing one or more of these moieties.

The sample solution can be any solution containing an analyte of interest. The invention is particularly useful for extraction and purification of biological molecules, hence the sample solution is often of biological origin, e.g., a cell lysate. In one embodiment of the invention the sample solution is a hybridoma cell culture supernatant.

In some embodiments, the microprocessor is external to the body of the pipettor, e.g., an external PC programmed to control a sample processing procedure. In some embodiments the piston is driven by a motor, e.g., a stepper motor.

The invention provides a pipettor (such as a multi-channel pipettor) suitable for acting as the pump in methods such as those described above. In some embodiments the pipettor comprises an electrically driven actuator. The electrically driven actuator can be controlled by a microprocessor, e.g., a programmable microprocessor. In various embodiments the microprocessor can be either internal or external to the pipettor body. In certain embodiments the microprocessor is programmed to pass a pre-selected volume of solution through the bed of medium at a pre-selected flow rate.

The low back pressure associated with certain columns of the invention results in some cases in the columns exhibiting characteristics not normally associated with conventional packed columns. For example, in some cases it has been observed that below a certain threshold pressure solvent does not flow through the column. This threshold pressure can be thought of as a “bubble point.” In conventional columns, the flow rate through the column generally increases from zero as a smooth function of the pressure at which the solvent is being pushed through the column. With many of the columns of the invention, a progressively increasing pressure will not result in any flow through the column until the threshold pressure is achieved. Once the threshold pressure is reached, the flow will start at a rate significantly greater than zero, i.e., there is no smooth increase in flow rate with pressure, but instead a sudden jump from zero to a relatively fast flow rate. Once the threshold pressure has been exceeded flow commences, the flow rate typically increases relatively smoothly with increasing pressure, as would be the case with conventional columns.

After the sample solution has been introduced into the bed and analyte allowed to adsorb, the sample solution is substantially evacuated from the bed, leaving the bound analyte. It is not necessary that all sample solution be evacuated from the bed, but diligence in removing the solution can improve the purity of the final product. An optional wash step between the adsorption and desorption steps can also improve the purity of the final product. Typically water or a buffer is used for the wash solution. The wash solution is preferably one that will, with a minimal desorption of the analyte of interest, remove excess matrix materials, lightly adsorbed or non-specifically adsorbed materials so that they do not come off in the elution cycle as contaminants. The wash cycle can include solvent or solvents having a specific pH, or containing components that promote removal of materials that interact lightly with the extraction phase. In some cases, several wash solvents might be used in succession to remove specific material, e.g., PBS followed by water. These cycles can be repeated as many times as necessary. In other cases, where light contamination can be tolerated, a wash cycle can be omitted. The wash solution can optionally be passed through the column more than one time, e.g., by being pumped back and forth through the bed using repeated aspirate/expel cycles.

The volume of desorption solvent used can be very small, approximating the interstitial volume of the bed of extraction medium. In certain embodiments of the invention the amount of desorption solvent used is less than 10-fold greater than the interstitial volume of the bed of extraction medium, more preferably less than 5-fold greater than the interstitial volume of the bed of extraction medium, still more preferably less than 3-fold greater than the interstitial volume of the bed of extraction medium, still more preferably less than 2-fold greater than the interstitial volume of the bed of extraction medium, and most preferably is equal to or less than the interstitial volume of the bed of extraction medium. For example, ranges of desorption solvent volumes appropriate for use with the invention can have a lower limit of 1%, 5%, 10%, 15%, 20%, 30%, 40%, 50%, 60%, 70%, 80%, 90%, 100%, 150%, 200% or 300% of the interstitial volume, and an upper limit of 50%, 100%, 200%, 300%, 400%, 500%, 500%, 600%, 700%, 800%, or 1000% of the interstitial volume, e.g., 10 to 200% of the interstitial volume, 20 to 100% of the interstitial volume, 10 to 50%, 100% to 500%, 200 to 1000%, etc., of the interstitial volume.

Alternatively, the volume of desorption solvent used can be quantified in terms of percent of bed volume (i.e., the total volume of the medium plus interstitial space) rather than percent of interstitial volume. For example, ranges of desorption solvent volumes appropriate for use with the invention can have a lower limit of 1%, 5%, 10%, 15%, 20%, 30%, 40%, 50%, 60%, 70%, 80%, 90%, 100%, 150%, 200% or 300% of the bed volume, and an upper limit of 50%, 100%, 200%, 300%, 400%, 500%, 500%, 600%, 700%, 800%, or 1000% of the bed volume, e.g., 10 to 200% of the bed volume, 20 to 100% of the bed volume 10 to 50%, 100% to 500%, 200 to 1000%, etc., of the bed volume.

In some embodiments of the invention, the amount of desorption solvent introduced into the column is less than 100 μL, less than 20 μL, less than 15 μL, less than 10 μL, less than 5 μL, or less than 1 uL. For example, ranges of desorption solvent volumes appropriate for use with the invention can have a lower limit of 0.1 μL, 0.2 μL, 0.3 μL, 0.5 μL, 1 μL, 2 μL, 3 μL, 5 μL, or 10 μL, and an upper limit of 2 μL, 3 μL, 5 μL, 10 μL, 15 μL, 20 μL, 30 μL, 50 μL, or 100 μL, e.g., in between 1 and 15 μL, 0.1 and 10 μL, or 0.1 and 2 μL.

The desorption solution can optionally be passed through the column more than one time by being pumped back and forth through the bed using repeated aspirate/expel cycles.

### Low Volume Elution of Biomolecules from Pipette Tip Columns.

In some embodiments of the invention it is desirable to elute the analyte in a very small volume of desorption solvent. In these embodiments, good recovery of the analyte is obtained due to the nature of the resin of the instant invention. The use of small volumes of desorption solution enables one to achieve high enrichment factors in the described methods. The term “enrichment factor” as used herein is defined as the ratio of the sample volume divided by the elution volume, assuming that there is no contribution of liquid coming from the dead volume. To the extent that the dead volume either dilutes the analytes or prevents complete adsorption, the enrichment factor is reduced. For example, if 1000 μL of sample solution is loaded onto the column and the bound analyte eluted in 10 μL of desorption solution, the calculated enrichment factor is 100. Note that the calculated enrichment factor is the maximum enrichment that can be achieved with complete capture and release of analyte. Actual achieved enrichments will typically lower due to the incomplete nature of most binding and release steps. Various embodiments of the invention can achieve ranges of enrichment factors having a lower limit of 1, 10, 100, or 1000, and an upper limit of 10, 100, 1000, 10,000 or 100,000.

In some embodiments of the invention, a particular eluent is chosen because it does not interact with residual ion exchange groups. For example, in the case of a Ni IMAC affinity resin, a low pH elution can protonate the His-tagged protein and/or chelating groups.

PhyTip columns made by PhyNexus, Inc. (San Jose) are pipette tip columns that work well with the methods of the invention. PhyTip columns are designed to use the minimum possible elution strength and elution volume to remove and recover the biomolecule from the column. This is accomplished by designing the PhyTip column to be very low dead volume so that the elution solvent can interact quickly and directly with the resin containing the analyte. The PhyTip column frits are very thin and have extremely low dead volume. In fact the thickness of a PhyTip column frit or screen is actually less than the average bead diameter of the beads being held in place by the frit. The thickness of the frit is 60 μm whereas the average bead diameter of a typical resin being held is 90 μm.

In addition, the column bed is located at the tip of the pipette tip body so that no dead volume is required to be filled before the elution solvent can contact the column bed. The bottom column frit is hydrophilic so that liquid is drawn up into the column upon contact and. The elution liquid travels directly into the bed so that the elution process can begin immediately.

Since the interstitial volume of the bed is approximately one third to one half of the bed volume, in theory, the elution volume may only have to be one third to one half of the bed volume. This volume added to the interstitial volume would double the total volume in the elution volume. For example, a 5 μL PhyTip column bed (in a 200 μL tip body) could only require 1.5 to 2.5 μL elution volume to remove material from the column. This volume added to the interstitial volume already present would give a total of 3.0 to 5.0 μL that could be expelled into a well upon removal of the liquid. (Of course, the elution volume is diluted so that has to be taken into consideration to make certain the concentration of the eluant is sufficient). In practice, a larger elution volume is used because it is difficult to pick up a 1.5 to 2.5 μL drop into a column and also the entire column interstitial volume cannot not be completely expelling into a well upon elution (resulting in protein loss). Still, it is possible to elute most bound material with a 10 μL or even a 5 μL size elution volume making PhyTip columns the most optimized columns for small volume elution.

With optimization of the column hardware, experiments were performed to elute materials in as small of volume as possible and with as small of elution concentration as possible. By performing experiments of recovery versus elution volume and recovery vs. elution strength a new phenomenon has been discovered that affects the elution volume required to recover material from a column. These experiments showed that no elution or very little elution occurs with the first addition of eluting solvent. A threshold must be reached before elution will start and complete elution is dependant upon adding more active reagent than can be calculated to be necessary. In other words, the mole recovery of a protein or other biomolecule is not directly proportional to the mole amount of eluting active reagent. It turns out that this effect is due to the residual groups remaining on the resin synthesis discussed above in the Extraction media section. Using the resins, columns and methods of the invention it is possible to get very high recovery of the eluted analyte in a very small volume without the use of harsh elution conditions (such as strong acid).

Sometimes in order to improve recovery it is desirable to pass the desorption solvent through the extraction bed multiple times, e.g., by repeatedly aspirating and discharging the desorption solvent through the extraction bed and lower end of the column. Step elutions can be performed to remove materials of interest in a sequential manner. Air may be introduced into the bed at this point (or at any other point in the procedure), but because of the need to control the movement of the liquid through the bed, it is not preferred except perhaps in the final expulsion of proteins.

The desorption solvent will vary depending upon the nature of the analyte and extraction medium. For example, where the analyte is a his-tagged protein and the extraction medium an IMAC resin, the desorption solution will contain imidazole or the like to release the protein from the resin. In some cases desorption is achieved by a change in pH or ionic strength, e.g., by using low pH or high ionic strength desorption solution. A suitable desorption solution can be arrived at using available knowledge by one of skill in the art.

Extraction columns and devices of the invention should be stored under conditions that preserve the integrity of the extraction medium. For example, columns containing agarose- or sepharose-based extraction medium should be stored under cold conditions (e.g., 4 degrees Celsius) and in the presence of 0.01 percent sodium azide or 20 percent ethanol. Prior to extraction, a conditioning step may be employed. This step is to ensure that the tip is in a uniform ready condition, and can involve treating with a solvent and/or removing excess liquid from the bed. If agarose or similar gel materials are used, the bed should be kept fully hydrated before use.

Often it is desirable to automate the method of the invention. For that purpose, the subject invention provides a device for performing the method comprising a column containing a packed bed of extraction medium, a pump attached to one end of said column, and an automated means for actuating the pump.

The automated means for actuating the pump can be controlled by software. This software controls the pump, and can be programmed to introduce desired liquids into a column, as well as to evacuating the liquid by the positive introduction of gas into the column if so desired.

In some embodiments the invention comprises methods for using pipette tip columns containing resins that lack residual ion exchange groups.

For example, in certain embodiments the invention provides a general method for passing liquid through a packed-bed pipette tip column comprising the steps of:
a) providing a first column comprising:
   i. a column body having an open upper end for communication with a pump, a first open lower end for the uptake and dispensing of fluid, and an open passageway between the upper and lower ends of the column body;
   ii. a bottom frit attached to and extending across the open passageway;
   iii. a top frit attached to and extending across the open passageway between the bottom frit and the open upper end of the column body, wherein the top frit, bottom frit, and surface of the passageway define a media chamber;
   iv. a first packed bed of medium positioned inside the media chamber;
   v. a first head space defined as the section of the open passageway between the open upper end and the top frit, wherein the head space comprises a gas having a first head pressure; and
   vi. a pump sealingly attached to the open upper end, where actuation of the pump affects the first head pressure, thereby causing fluid to be drawn into or expelled from the bed of medium;
b) contacting said first open lower end with a first liquid;
c) actuating the pump to draw the first liquid into the first open lower end and through the first packed bed of medium; and
d) actuating the pump to expel at least some of the first liquid through the first packed bed of medium and out of the first open lower end.

In certain embodiments, the invention further comprises the following steps subsequent to step (d):
e) contacting said first open lower end with a second liquid, which is optionally the same as the first liquid;
f) actuating the pump to draw second liquid into the first open lower end and through the first packed bed of medium; and
g) actuating the pump to expel at least some of the second liquid through the first packed bed of medium and out of the first open lower end.

In certain embodiments, the first head pressure of the first column is adjusted between steps (d) and (f) to render the head pressure closer to a reference pressure. For example, in certain embodiments the first head pressure of the first column is adjusted between steps (d) and (f) to render the first head pressure substantially equal to a reference head pressure. Likewise, in certain embodiments the reference head pressure is predetermined and/or is the head pressure of the first column prior to step (c).

In a number of embodiments, the above-described method further comprises the steps of:
h) providing a second column comprising:
   i. a column body having an open upper end for communication with a pump, a second open lower end for the uptake and dispensing of fluid, and an open passageway between the upper and lower ends of the column body;
   ii. a bottom frit attached to and extending across the open passageway;
   iii. a top frit attached to and extending across the open passageway between the bottom frit and the open upper end of the column body, wherein the top frit, bottom frit, and surface of the passageway define a media chamber;
   iv. a second packed bed of medium positioned inside the media chamber;
   v. a second head space defined as the section of the open passageway between the open upper end and the top frit, wherein the head space comprises a gas having a second head pressure; and
   vi. a pump sealingly attached to the second open upper end, where actuation of the pump affects the second head pressure, thereby causing fluid to be drawn into or expelled from the second packed bed of medium;
i) contacting said second open lower end with a third liquid, which is optionally the same as the first liquid;
j) actuating the pump to draw the third liquid into the second open lower end and through the second packed bed of medium;
k) actuating the pump to expel at least some of the third liquid through the second packed bed of medium and out of the second open lower end.
l) contacting said second open lower end with a fourth liquid, which is optionally the same as the third liquid;
m) actuating the pump to draw fourth liquid into the second open lower end and through the second packed bed of medium; and
n) actuating the pump to expel at least some of the fourth liquid through the second packed bed of medium and out of the second open lower end,
   wherein the head pressure of the second column is adjusted between steps (k) and (m) to render the head pressure closer to a reference pressure.

In the foregoing methods, steps (b) through (g) can be performed prior to steps (i) through (n). Alternatively, steps (b) through (g) can be performed concurrently and in parallel with steps (i) through (n). In either case, the reference head pressure can be the head pressure of the first column immediately prior to the commencement of step (f). The pump can be a multi-channel pipettor and the first column can be attached to a first channel of the multi-channel pipettor and the second column can be attached to a second channel of the multi-channel pipettor. Between steps (d) and (f) the first head pressure can be adjusted to render the first and second head pressures more uniform. In some cases the method is applied concurrently and in parallel to at least six pipette tip columns sealingly attached to said multi-channel pipettor, wherein each pipette tip column comprises a head space having a head pressure, and wherein the head pressures of the at least six pipette tip columns are adjusted to render the head pressures more uniform.

If the sample volume is larger than the interstitial volume of the bed, sample is drawn through the bed and into the column body above the upper frit. The sample solution is then expelled back into the sample container. In some embodiments, the process of drawing sample through the bed and back out into the sample container is performed two or more times, each of which results in the passage of the sample through the bed twice. As discussed elsewhere herein, analyte adsorption can in some cases be improved by using a slower flow rate and/or by increasing the number of passages of sample through the extraction medium.

The sample container is then removed and replaced with a similar container holding wash solution (e.g., in the case of an immobilized metal extraction, 5 mM imidazole in PBS), and the wash solution is pumped back and forth through the extraction bed (as was the case with the sample). The wash step can be repeated one or more times with additional volumes of wash solution. A series of two or more different wash solutions can optionally be employed, e.g., PBS followed by water.

Next, an aliquot of desorption solvent is drawn through the bed of extraction medium (e.g., 15 μL of a low pH solvent would be typical for elution of protein off an immobilized metal column having a bed volume of about 10 μL). The elution solution can be manipulated back and forth through the bed multiple times by repeated cycles of aspirating and expelling the solution through the column. The elution cycle is completed by ejecting the desorption solution back into the sample vial. The elution process can be repeated, in some cases allowing for improved sample recovery.

The above-described extraction process can be automated, for example by using software to program the computer controller to control the pumping, e.g., the volumes, flow rates, delays, and number of cycles.

In some embodiments, the invention provides a multiplexed extraction system comprising a plurality of extraction columns of the invention, e.g., low dead volume pipette tip columns having small beds of packed gel resins. The system can be automated or manually operated. The system can include a pump or pump in operative engagement with the extraction columns, useful for pumping fluid through the columns in a multiplex fashion, i.e., concurrently. In some embodiments, each column is addressable. The term “addressable” refers to the ability of the fluid manipulation mechanism, e.g., the pumps, to individually address each column. An addressable column is one in which the flow of fluid through the column can be controlled independently from the flow through any other column which may be operated in parallel. In practice, this means that the pumping means in at least one of the extraction steps is in contact and control of each individual column independent of all the other columns. For example, when syringe pumps are used, i.e., pumps capable of manipulating fluid within the column by the application of positive or negative pressure, then separate syringes are used at each column, as opposed to a single vacuum attached to multiple syringes. Because the columns are addressable, a controlled amount of liquid can be accurately manipulated in each column. In a non-addressable system, such as where a single pump is applied to multiple columns, the liquid handling can be less precise. For example, if the back pressure differs between multiplexed columns, then the amount of liquid entering each column and/or the flow rate can vary substantially in a non-addressable system. Various embodiments of the invention can also include samples racks, instrumentation for controlling fluid flow, e.g., for pump control, etc. The controller can be manually operated or operated by means of a computer. The computerized control is typically driven by the appropriate software, which can be programmable, e.g., by means of user-defined scripts.

The invention also provides software for implementing the methods of the invention. For example, the software can be programmed to control manipulation of solutions and addressing of columns into sample vials, collection vials, for spotting or introduction into some analytical device for further processing.

The invention also includes kits comprising one or more reagents and/or articles for use in a process relating to solid-phase extraction, e.g., buffers, standards, solutions, columns, sample containers, etc.

### Purification of Classes of Proteins

Extraction columns can be used to purify entire classes of proteins on the basis of highly conserved motifs within their structure, whereby an affinity binding agent is used that reversibly binds to the conserved motif. For example, it is possible to immobilize particular nucleotides on the extraction medium. These nucleotides include adenosine 5′-triphosphate (ATP), adenosine 5′-diphosphate (ADP), adenosine 5′-monophosphate (AMP), nicotinamide adenine dinucleotide (NAD), or nicotinamide adenine dinucleotide phosphate (NADP). These nucleotides can be used for the purification of enzymes that are dependent upon these nucleotides such as kinases, phosphatases, heat shock proteins and dehydrogenases, to name a few.

There are other affinity groups that can be immobilized on the extraction medium for purification of protein classes. Lectins can be employed for the purification of glycoproteins. Concanavilin A (Con A) and lentil lectin can be immobilized for the purification of glycoproteins and membrane proteins, and wheat germ lectin can be used for the purification of glycoproteins and cells (especially T-cell lymphocytes). Though it is not a lectin, the small molecule phenylboronic acid can also be immobilized and used for purification of glycoproteins.

It is also possible to immobilize heparin, which is useful for the purification of DNA-binding proteins (e.g. RNA polymerase I, II and III, DNA polymerase, DNA ligase). In addition, immobilized heparin can be used for purification of various coagulation proteins (e.g. antithrombin III, Factor VII, Factor IX, Factor XI, Factor XII and XIIa, thrombin), other plasma proteins (e.g. properdin, BetaIH, Fibronectin, Lipases), lipoproteins (e.g. VLDL, LDL, VLDL apoprotein, HOLP, to name a few), and other proteins (platelet factor 4, hepatitis B surface antigen, hyaluronidase). These types of proteins are often blood and/or plasma borne. Since there are many efforts underway to rapidly profile the levels of these types of proteins by technologies such as protein chips, the performance of these chips will be enhanced by performing an initial purification and enrichment of the targets prior to protein chip analysis.

It is also possible to attach protein interaction domains to extraction medium for purification of those proteins that are meant to interact with that domain. One interaction domain that can be immobilized on the extraction medium is the Src-homology 2 (SH2) domain that binds to specific phosphotyrosine-containing peptide motifs within various proteins. The SH2 domain has previously been immobilized on a resin and used as an affinity reagent for performing affinity chromatography/mass spectrometry experiments for investigating in vitro phosphorylation of epidermal growth factor receptor (EGFR) (see Christian Lombardo, et al., Biochemistry, 34:16456 (1995)). Other than the SH2 domain, other protein interaction domains can be immobilized for the purposes of purifying those proteins that possess their recognition domains. Many of these protein interaction domains have been described (see Tony Pawson, Protein Interaction Domains, Cell Signaling Technology Catalog, 264-279 (2002)) for additional examples of these protein interaction domains).

As another class-specific affinity ligand, benzamidine can be immobilized on the extraction medium for purification of serine proteases. The dye ligand Procion Red HE-3B can be immobilized for the purification of dehydrogenases, reductases and interferon, to name a few.

In another example, synthetic peptides, peptide analogs and/or peptide derivatives can be used to purify proteins, classes of proteins and other biomolecules that specifically recognize peptides. For example, certain classes of proteases recognize specific sequences, and classes of proteases can be purified based on their recognition of a particular peptide-based affinity binding agent.

In one embodiment, a series of two or more desorption solvents is used sequentially, and the eluent is monitored to determine which protein constituents come off at a particular solvent. In this way it is possible to assess the strength and nature of interactions in the complex. For example, if a series of desorption solvents of increasing strength is used (e.g., increasing ionic strength, decreasing polarity, changing pH, change in ionic composition, etc.), then the more loosely bound proteins or sub-complexes will elute first, with more tightly bound complexes eluting only as the strength of the desorption solvent is increased.

In some embodiments, at least one of the desorption solutions used contains an agent that effects ionic interactions. The agent can be a molecule that participates in a specific interaction between two or more protein constituents of a multi-protein complex, e.g., Mg-ATP promotes the interaction and mutual binding of certain protein cognates. Other agents that can affect protein interactions are denaturants such as urea, guanidinium chloride, and isothiocyanate, detergents such as triton X-100, chelating groups such as EDTA, etc.

In other sets of experiments, the integrity of a protein complex can be probed through modifications (e.g., post-translational or mutations) in one or more of the proteins. Using the methods described herein the effect of the modification upon the stability or other properties of the complex can be determined.

### Recovery of Native Proteins

In some embodiments, the extraction devices and methods of the invention are used to purify proteins that are functional, active and/or in their native state, i.e., non-denatured. This is accomplished by performing the extraction process under non-denaturing conditions. Non-denaturing conditions encompasses the entire protein extraction process, including the sample solution, the wash solution (if used), the desorption solution, the extraction phase, and the conditions under which the extraction is accomplished. General parameters that influence protein stability are well known in the art, and include temperature (usually lower temperatures are preferred), pH, ionic strength, the use of reducing agents, surfactants, elimination of protease activity, protection from physical shearing or disruption, radiation, etc. The particular conditions most suited for a particular protein, class of proteins, or protein-containing composition vary somewhat from protein to protein.

Examples of protein analytes that can be beneficially processed by the technology described herein include antibodies (e.g., IgG, IgY, etc.); general affinity proteins, (e.g., scFvs, Fabs, affibodies, peptides, etc.); nucleic acids aptamers and photoaptamers as affinity molecules, and other proteins to be screened for undetermined affinity characteristics (e.g., protein libraries from model organisms). The technology is particularly useful when applied to preparation of protein samples for global proteomic analysis, for example in conjunction with the technology of Protometrix Inc. (Branford, Conn.). See, for example, Zhu et al. “Global analysis of protein activities using proteome chips (2001) Science 293(5537): 2101-05; Zhu et al., “Analysis of yeast protein kinases using protein chips” (2000) Nature Genetics 26:1-7; and Michaud and Snyder “Proteomic approaches for the global analysis of proteins” (2002) BioTechniques 33:1308-16.

### Multiplexing

In some embodiments of the invention a plurality of columns is run in a parallel fashion, e.g., multiplexed. This allows for the simultaneous, parallel processing of multiple samples. Multiplexing can be accomplished by use of the columns with a pipetting robot or liquid handling system such as the MEA Personal Purification System™ from PhyNexus, Inc., San Jose. The MEA operating Manual is available from the PhyNexus website (http://www.phynexus.com/). Other liquid handling systems that can be used with the methods of the instant invention include those manufactured by Zymark (e.g., the SciClone sample handler), Tecan (e.g., the Genesis NPS, Aquarius or TeMo) or Cartesian Dispensing (e.g., the Honeybee benchtop system), Packard (e.g., the MiniTrak5, Evolution, Platetrack, or Apricot), Beckman (e.g., the FX-96) and Matrix (e.g., the Plate Mate 2 or SerialMate).

When using a multiplexed system, it may be desirable to program delays into the software controlling the protocol. When several columns are operated in parallel, each column may have a slightly different back pressure. As a result, the flow rate of a liquid through each column may vary when vacuum or pressure is applied to the columns. One means of compensating for the different flow rates is the incorporation of delays to equalize the vacuum or pressure and thus equalize the total amount of liquid dispensed or aspirated. Pauses can be used at any time during the protocol, e.g. while aspirating, dispensing, or between an aspiration and a dispense step.

In one example of a multiplexing procedure, 12 wells of a 96-well plate containing a sample, e.g., 0.5 ml of serum. Sample solution is drawn into 8 pipette-tip columns attached to the MEA Personal Purification System. Control of the liquid in the column is optionally bidirectional. If the sample volume is larger than the interstitial volume of the bed, sample is drawn through the bed and into the column body above the upper frit. The sample solution is then expelled back into the Eppendorf tube. In some embodiments, the process of drawing sample through the bed and back out into the sample container is performed two or more times, each of which results in the passage of the sample through the bed twice. As discussed elsewhere herein, analyte adsorption can in some cases be improved by using a slower flow rate and/or by increasing the number of passages of sample through the medium.

Next, the pipette-tip columns are washed. A 96-well plate holding a wash solution (e.g., in the case of a preparation utilizing an immobilized metal, PBS or similar buffer) is placed on the deck of the MEA instrument, and the wash solution is aspirated and dispensed back and forth through the extraction medium bed (as was the case with the sample). In some embodiments, the wash solution is not aspirated and dispensed repeatedly, but rather only once. In some embodiments, the wash step can be repeated one or more times with additional volumes of wash solution. A series of two or more different wash solutions can optionally be employed, e.g., PBS or buffer followed by water.

Finally, the analyte is eluted from pipette-tip columns. A 96-well plate holding a desorption solution is placed in the desired position on the deck of the MEA. The desorption solution is aspirated

The above-described preparation process is automated, for example by using software to program the computer controller that in turn, controls an automated liquid handling system such as the MEA Personal Purification System™ multi-channel pipettor, e.g., the volumes, flow rates, delays, and number of cycles. The software is programmable, e.g., by means of user-defined scripts. In some embodiments the invention provides software for implementing the methods of the invention. For example, the software can be programmed to control manipulation of solutions and addressing of columns into sample vials, collection vials, for spotting or introduction into some analytical device for further processing.

In some embodiments, the invention provides a multiplexed preparation system comprising a plurality of columns of the invention, e.g., pipette tip columns having beds of resins synthesized as described above in the Extraction media section.

In some embodiments, the invention also includes kits comprising one or more reagents and/or articles for use in a process relating to solid-phase extraction, e.g., buffers, standards, solutions, columns, plates, sample containers, etc.

All publications and patent applications mentioned in this specification are herein incorporated by reference to the same extent as if each individual publication or patent application was specifically and individually indicated to be incorporated by reference.

Having now generally described the invention, the same will be more readily understood through reference to the following examples, which are provided by way of illustration, and are not intended to be limiting of the present invention, unless so specified.

### EXAMPLES

The following preparations and examples are given to enable those skilled in the art to more clearly understand and practice the present invention. They should not be construed as limiting the scope of the invention, but merely as being illustrative and representative thereof.

### Example 1

### Evaluation of a 10 μL Bed Volume Pipette Tip Column Containing Two Different Protein A Resins Containing Residual Anion Exchange Sites

In this example, the performance of 10 μL bed volume pipette tip columns (manufactured from 1 mL Rainin pipette tips) containing two different Protein A resins were evaluated. The resins under consideration consist of purified recombinant protein A covalently coupled through multi-point attachment via reductive amidation to 6% highly cross-linked agarose beads (RepliGen Corporation, IPA-400HC; PN: 10-2500-02) or to 4% cross-linked sepharose beads (GE). It was previously determined that these resins contain one residual anion site for each Protein A site present. The samples tested consisted of 15 μg mFITC-MAb (Fitzgerald, Inc. Cat # 10-F50, mouse IgG₂ₐ) in 0.5 ml of PBS or PBS containing 5 mg BSA (10 mg/ml or 1% m/v BSA).

An ME-100 multiplexing extraction system (Phynexus, Inc.) was used for this extraction. The ME-100 is comprised of ten 1-ml syringes that are controlled in parallel by a pump that is driven by software. The 10 μL bed volume pipette tip columns described above were attached to the syringes. The 0.5 mL samples were provided in 1.5 ml Eppendorf tubes and positioned in the sample rack, which was raised so that the tip of the columns made contact with the sample. During the load cycle, 2 or 5 aspirate/expel cycles were employed (depending upon the test), the volume drawn or ejected programmed at 0.6 ml @ 0.25 ml/min.

After loading, the extraction beds were washed with 0.5 mL PBS for 2-4 aspirate/expel cycles, volume programmed at 0.5 ml @ 0.5 ml/min, or 1 wash with 1 ml PBS, volume programmed at 1.0 mL @ 0.5 mL/min followed by final wash with 0.5 mL H₂O. No differences were measured due to the number and volume of wash conditions.

The elution cycle involved 4 aspirate/expel cycles, volume programmed at 0.1-0.15 mL @ 1 ml/min (15 μL elution buffer, 111 mM NaH₂PO₄ in 14.8 mM H₃PO₄, pH 3.0).

To quantify the IgG recovered in the procedure and to analyze its purity, 13 μL of the 15 μL elution volume was reacted with freshly prepared 13 μL of 10 mg/ml TCEP (final volume=26 μL and [TCEP]=17.5 mM) at room temperature for ˜16 hours. 20 μL out of above 26 μl reduced IgG₂ₐ was injected into a non-porous polystyrene divinylbenzene reverse phase (C-18) column using an HP 1050 HPLC system. A gradient of 25% to 75% between solvent A which is 0.1% TFA in water and solvent B which is 0.1% TFA in ACN was used for 5 minutes. Detection: UV at 214 and 280 nm. There are two major IgG₂ₐ peaks having similar intensities as shown in the data below, which eluted around 3.17 and 3.3 min. Area under these two peaks was integrated from (3.13-3.5) min in each case and corresponding mAU was recorded at 214 nm. Since the majority of material eluted in the first fraction, only first elution (15 μl) percent recovery was calculated. TCEP-treated IgG₂ₐ standards (injected amount 1.08, 2.16, 4.32, 6.48 and 8.64 μg of FITC-MAb, obtained from Fitzgerald, Inc) under identical reaction condition was loaded into the column and used as a standard curve for recovery calculation.

The summary data were corrected for the fraction of the sample analyzed. The data shown below from these experiments indicate that_IgG purification using the Protein A extraction columns was highly selective. A 333-fold excess of BSA can quantitatively be removed in a very fast process.

The effectiveness of the hydrogen cation in the elution buffer to be able to elute the IgG from the resin is decreased because of the presence of residual anion exchange sites. Hydrogen cation is needed not only to elute the IgG from the Protein A site but also combines with the residual anion exchange sites. In this example there is a one to one ratio of anion exchange sites and protein A functional group. Hydrogen cation is taken up by these residual sites, thus decreasing the effectiveness of the elution buffer. Either a higher volume or higher concentration of buffer is for elution than if the residual sites where not present.

### Recoveries from Selectivity Assay (Determined by HPLC)

| Amersham | | Repligen |
|---|---|---|
| Recovery | Experimental Procedure | Recovery |
| 49% | 15 μg IgG₂ₐ/0.5 ml PBS (2 cycles loading) | 43% |
| 64% | 15 μg IgG₂ₐ/0.5 ml PBS + 5 mg BSA (2 cycles | 56% |
| | loading) | |
| 66% | 15 μg IgG₂ₐ/0.5 ml PBS + 5 mg BSA (5 cycles | 62% |
| | loading) | |

### Example 2

### Evaluation of a 10 μL Bed Volume Pipette Tip Column Containing a Protein A Resin with No Residual Anion Exchange Sites

In this example, everything is performed the same as in Example 1 except the resin is prepared with a synthesis procedure in which a divinylsulfone reagent is used to attach the recombinant Protein A to the agarose resin. First, the agarose resin is activated with vinyl sulfone which reacts with the hydroxyl group of the agarose resin leaving a reactive group on the opposite end. Then Protein A is added to the activated substrate with reacts to the divinyl sulfone activated agarose.

Because of the reduction in amine ion exchange sites, the same recovery elution is carried out with 10-50% less elution volume or 10-50% of the amount of hydrogen cation that was required in Example 1.

While the invention has been described in connection with specific embodiments thereof, it will be understood that it is capable of further modifications and this application is intended to cover and variations, uses, or adaptations of the invention that follow, in general, the principles of the invention, including such departures from the present disclosure as come within known or customary practice within the art to which the invention pertains and as may be applied to the essential features hereinbefore set forth. Moreover, the fact that certain aspects of the invention are pointed out as preferred embodiments is not intended to in any way limit the invention to such preferred embodiments.

We claim:

1. A method for purifying a protein analyte from a sample solution by solid phase extraction comprising:
(a) providing a pipette tip column comprised of
a packed bed of gel resin, wherein the packed bed of gel resin is comprised of agarose or sepharose, and wherein the gel resin is further comprised of an affinity group having an affinity for the protein analyte, and
wherein said gel resin lacks residual ion exchange groups;
(b) passing a sample solution through the pipette tip column;
(c) optionally passing a wash solution through the pipette tip column; and
(d) eluting the protein analyte by passing a low pH desorption solution through the pipette tip column, wherein the low pH desorption solution is comprised of hydrogen cations, wherein the hydrogen cations in the desorption solution dissociate the protein analyte from the affinity group, and wherein the protein analyte is an active protein.

2. The method of claim 1 wherein step (b), step (c) or step (d) is performed more than one time.

3. The method of claim 1 wherein the ion exchange groups are amines.

4. The method of claim 1 wherein the affinity group is proA, proG, proL or an immobilized metal.

5. A method for purifying a protein analyte from a sample solution by solid phase extraction comprising:
(a) providing a pipette tip column comprised of
a packed bed of gel resin, wherein the gel resin is comprised of agarose or sepharose, and wherein the gel resin is further comprised of a proA or proG affinity group, and
wherein the gel resin lacks residual amines;
(b) passing a sample solution through the pipette tip column;
(c) optionally passing a wash solution through the pipette tip column; and
(d) eluting the protein analyte by passing a low pH desorption solution through the pipette tip column, wherein the low pH desorption solution is comprised of hydrogen cations, and wherein the hydrogen cations in the desorption solution dissociate the protein analyte from the affinity group, and wherein the protein analyte is an active protein.

6. The method of claim 5 wherein step (b), step (c) or step (d) is performed more than one time.
